(19) United States
(12) Patent Application Publication
(10) Pub. No.: US 2008/0269545 A1
(43) Pub. Date: Oct. 30, 2008

(54) Dreamstate programming method and device

(75) Inventor: Brent Ellis Logan, Mount Vernon, WA (US)

Correspondence Address: Brent E. Logan, 1100 Nez Perce Drive, Mount Vernon, WA, 98273, US

(73) Assignee: Brent Ellis Logan, Mount Vernon, WA (US)

(21) Appl. No.: 12/148,865

(22) Filed: Apr. 23, 2008

Related U.S. Application Data
(60) Provisional application No. 60/926,529, filed on Apr. 27, 2007.

(51) Int. Cl. A61M 21/00

(52) U.S. Cl. 600/27

(57) ABSTRACT

Dreaming has been found to occur among mammals almost continuously throughout sleep, and can be influenced by environmental stimuli while taking place, particularly with humans by utilizing recordings of short, simple, and repeated questions in the sleeper's own voice, then played back at preset intervals during the sleepstate; this invention incorporates these factors as a methodology to enhance the homeostatic nature of dreams, more specifically focusing them for the sleeper's immediate and long-term wellbeing. The device employs sonic technology in various formats as a novel governor of nocturnal or naptime information-processing by the brain, permitting the safe and effective resolution of immediate issues as well as concerns from even the distant past; it is contraindicated as a diagnostic or therapeutic aid, without the intent of medical application.

### CROSS-REFERENCE TO RELATED APPLICATIONS

This application claims the benefit of U.S. Provisional Patent Application 60/926,529 having a filing date of Apr. 27, 2007, the primary contents of which are hereby incorporated by reference.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

Over the last several decades cognition and behavior have been undergoing major redefinitions, with these explanations generally more complex than before. Science now understands that the influences which lead us to arrive at beneficial ideas or lifestyles include numerous sources not previously considered. Especially effective is the power of suggestion applied through novel means. This invention proposes a unique approach based upon recent sleep research.

The standard interpretation of sleeping recognizes both its physiological and psychological value, without which vertebrates—notably mammals—rapidly decline in functionality. Lately, extending our earlier knowledge, dreams are seen as crucial for more comprehensive maintenance—reviewing unresolved issues, anticipating weaknesses, rehearsing scenarios against perceived threats, providing solutions to personal or even cultural challenges, sometimes inspiring innovation in the arts or sciences.

Two major discoveries are particularly relevant. Robert L. Van de Castle, in Our Dreaming Mind (1994), summarizes the first, referring to research in the 1960s: “A new conceptualization of the mind's activity during sleep was now required. The previous model . . . held that dreams emerged abruptly at regular intervals during sleep and were nonexistent in the interim periods. The non-REM [rapid eye movement] and sleep onset findings, however, indicated that there is no period during sleep in which our mind is ‘blank’; some kind of mental activity is always occurring.” He then reviews evidence that such subconscious industry includes continuous dreaming at various levels, this conclusion underscored by Mark Solms, whose 2003 study, “Dreaming and REM Sleep are Controlled by Different Brain Mechanisms,” presents new technology's confirmation that REM sleep is not the exclusive province of dreams but sometimes contains no incidence of them. Earlier, David Foulkes—following his research in the 1960s—and Corrado Cavallero had written (1993), “there almost certainly is REM sleep without dreaming and . . . there is certainly dreaming without REM sleep.”

In 2003 Antti Revonsuo postulated threat simulation theory (TST): “Dream content shows a significant bias toward representing threatening elements . . . ” Out of prehistoric times a genetically encoded survival mechanism had evolved in the dream process, responding to the subject's critical issues, particularly where fear, anxiety, pain, loss, frustration, grief, and regret were concerned; this preemptive defense system, rehearsing dangers already encountered—at least to some extent—across an endless spectrum of often bizarre scapes and situations peopled with familiars or strangers, acts as a supportive counterpart to innate biological preservatives, namely, a mental immune system which fortifies against possible cognitive, psychological, emotional, and behavioral hazards, also adding nonphysical but equally curative scar tissue to injuries of this nature in the recent or remote past. Individuals possessing such an advantage would therefore be more likely to thrive, placing them preeminently on the Darwinian fitness scale for preferable mate selection, a classic example of evolutionary psychology's impact upon genealogy.

Further, it has been known since the work of Hervey de Saint-Denys in the mid-1800s that not only does the brain record surrounding events unperceived by the sleeper yet retrievable under hypnosis, but that multisensory stimuli alter dream content; in 1969 Fernandez confirmed electroencephalographic detection of auditory stimuli by the brain during sleep. Given this revised explanation for dreaming as an essential reenactment of previous problems in order to avoid their recurrence, along with a receptivity to subliminal suggestion, has led the applicant to develop technology which would focus the specific deficits of an individual upon these natural tools—for potentially profound and permanent mental or behavioral benefits.

### BRIEF SUMMARY OF THE INVENTION

Because dreaming has been increasingly identified as a homeostatic survival process concurrent with most sleep, and this innate means receptive to environmental influence, it is herewith proposed that dream content can be influenced to more effectively address particular requirements of the dreamer. Essentially, this innovation relies upon the dream's plasticity—like a developmental window—to place no value judgement upon its source, providing the signal is appropriately formatted: If the dreamer can reinforce their wishes upon those mechanisms which shape a dream, then it will respond by drawing upon the much greater reservoir of information and ability which constitutes the subconscious mind yet is not accessible during our waking lives.

How to set about “advising” one's dreams? As Castaldo and Holzman verified in 1969, prompts are most successful if spoken by the dreamer, their own voice—its tone, pitch, syntax, and rhythm—best recognized by that brain which initiates the very vocalization; Van de Castle's 1994 statement reaffirms this: “the dreamer's own voice noticeably increases dream activity and assertiveness.” But if the dreamer is asleep, what shall be the source of such persuasion?

Several years ago product claims were being made for nocturnal learning tapes—intoning foreign languages, self help messages, etc.—but despite some initial success this approach finally succumbed to robust criticism. This innovation operates quite differently: By repeatedly influencing the dream process with a short, simple question—in the sleeper's own voice, that most intimate link to the dreamstate—for only a brief period, the morphological dynamic of subliminal consciousness is permitted to shape its response accordingly, henced steered by internalized cues toward the pertinent topic and not distracted with conflicting input; throughout the night these reminders continue routing the genetically mandated dreamstate toward that single issue to which it will react in constantly shifting scenarios with diverse characters—usually known to the dreamer—like facets on a gemstone reflecting one lightsource in various aspects.

When morning arrives, the individual will have had a key query addressed in spatial and temporal settings relevant to that person's background: Their night's dreams examined the vital matter through layers of meaning that if not evident at once will surface when triggered by subsequent events—those identification points inflecting dreams because drawn from the dreamer's experiences hence likely to take place again in daily life if not thought (people, places, situations, etc.). Should resolution of the question not become evident by the following night, that same topic can be repeated; since the sleep dynamic exists to function on the dreamer's behalf, its comprehensive resources will bear upon the issue until an optimal answer surfaces.

By significant modification of a digital sound recorder/player, the dreamer's voice becomes the sleeptime trigger articulated over preselected intervals for permitting their dream to respond in kind. Rather than a directive which might be resisted by the dreamer's indigenous defenses—those psychodynamic features which assure existential balance—the desired direction can be shaped by a clear, abbreviated, and recurrent question, samples included with the device's instructions: “How will I meet the deadline?” “Should I change jobs?” “Where should I live?” “What is the solution to [fill in problem]?” “How might I lose weight [stop an addiction, etc.]?” While answers will emerge only from the dreamer's mind (comprising their intelligence, imagination, experience, emotion)—thus queries about winning lottery numbers or length of life are meaningless—it can be readily surmised that such applications should noticeably reduce dependency upon traditional resources. Corporate usage might have team members separately engage the technology with a common challenge.

Whispered several times, or by incorporating a volume control in the unit, the recorded question is broadcast over a short sequence from the player's speaker, an in-the-ear speaker, or pillow speaker (utilization of these options dependent upon the dreamer's preference or circumstance—such as the presence of another sleeper) at preset intervals throughout the night or naptime, conforming to whichever periodicity a user discovers most effective; because REM sleep has now been found not exclusively indicative of dreaming (Solms, 2003), the verbal cues may be set for playing at any point. Adjunctive components to the technology would include an AC power connector, and, if portability is desired, batteries.

As listed in the U.S. Patent Documents above, prior art involves waking the sleeper or employing REM detection capability, both elements rendered irrelevant by the Solms 2003 finding noted above: If dreams occur across the entire sleepstate, with REM periods not signifying their sole presence—even at times containing an absence thereof—neither conscious recall or REM focus is requisite to determine dream activity. Moreover, Raynie, et al. specifies lucid dreaming as that approach's functional means; but, irrespective of various techniques, the realization that one is dreaming by no means registers universally—and for many if not most remains impossible. Beyond this serious limitation, lucid dreaming raises a profound question about its ability to allow issues in the subconscious their deeper voice, since the sleeper's active participation in the dreamstate may be a constraint against the natural surfacing of distant or traumatic material. However, influencing dream content at all levels can be effortlessly achieved by the employment of technology which permits access to the full span of sleep, whether at night or during naps, achieving this goal with interrogative rather than declamatory sonic triggers available at self-selected intervals, and not relying upon the interruption of somnolence or wearing an often uncomfortable retinal light-stimulation apparatus on the head.

### BRIEF DESCRIPTION OF THE DRAWING

The unique features that are characteristic of the present invention are set forth in the appended claims. However, the preferred embodiments of the invention, together with further objects and attendant advantages, are best understood by reference to the following detailed description taken in connection with the accompanying drawing in which:

FIG. 1 is a ¾ frontal perspective view of the device as it might reside near a sleeper's head, the controls within reach, microphone opening at an effective distance, and speaker audible.

### DETAILED DESCRIPTION OF THE INVENTION

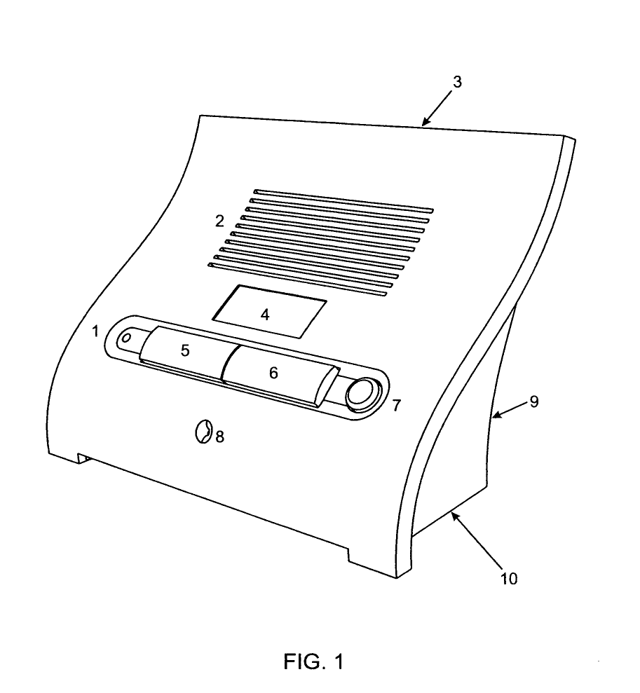

Referring to FIG. 1, one embodiment of the present innovation in a ¾ frontal perspective illustrates as if proximate to a sleeper's head an input jack, 1, for an optional earphone or pillow speaker connected to the device's sonic output circuitry, with 2 representing a loudspeaker face linked for message retrieval to the same digital memory source; 3 depicts the unit's contoured housing constructed of any safe and liquid-impervious material; 4 shows a liquid crystal display for numerical readout of the playback's timer, whose digits are increased by button 5, and decreased with button 6; 7 is the device's on/off button; 8 locates the microphone pickup leading to a microchip for digital message storage; 9 points to the location of an AC power cord; and 10 identifies the cover for battery placement.

Those versed in the art will appreciate that changes and emendations can be made to the embodiments and descriptions herein without departing from the spirit of the present innovation. All such alternatives and alterations are intended to be subsumed by the claims stated herein.

### REFERENCES CITED

### U.S. Patent Documents

U.S. Pat. No. 4,735,199 April 1988 DiLullo
U.S. Pat. No. 4,863,259 September 1989 Schneider, et al.
U.S. Pat. No. 5,507,716 April 1996 LaBerge, et al.
U.S. Pat. No. 5,551,879 September 1996 Raynie, et al.
U.S. Pat. No. 6,540,664 April 2003 Blair

### Other References

Castaldo, M. and Holzman, P., The effects of hearing one's own voice on dream content: a replication, Journal of Nervous and Mental Disease, 1969, 148:74-82.
Fernandez, H., Evoked K-complexes in the early stages of sleep and their relation to visual images, American Journal of EEG Technology, 1969, 9:18-21.
Foulkes, D., Dream reports from different stages of sleep, Journal of Abnormal and Social Psychology, 1962, 65:14-25.
Foulkes, David, and Cavallero, Corrado, editors, Dreaming as Cognition, Upper Saddle River, N.J., Prentice-Hall, 1993, page 9.
Hobson, J. Allan, The Dreaming Brain, New York, BasicBooks, 1988.
Hobson, J. A., Pace-Schoft, E. F., and Stickgold, R., Dreaming and the brain: toward a cognitive neuroscience of conscious states, in Sleep and Dreaming: Scientific Advances and Reconsiderations, Edward F. Pace-Schott, Mark Solms, Mark Blagrove, and Steven Harnad, editors, Cambridge, UK, Cambridge University Press, 2003, pages 1-50.
Kaiser, V. A., The effects of an auditory subliminal message upon the production of images and dreams, Journal of Nervous and Mental Disease, 1986, 174:397-407.
Revonsuo, A., and Valli, K., Dreaming and consciousness: testing the threat simulation theory of the function of dreaming, Psyche, October 2000, 6.
Revonsuo, A., The reinterpretation of dreams: an evolutionary hypothesis of the function of dreaming, in Sleep and Dreaming: Scientific Advances and Reconsiderations, Edward F. Pace-Schott, Mark Solms, Mark Blagrove, and Steven Harnad, editors, Cambridge, UK, Cambridge University Press, 2003, pages 85-109.
Saint-Denys, Hervey de, Dreams and How to Guide Them, London Duckworth, 1982.
Snyder, F., Toward an evolutionary theory of dreaming, American Journal of Psychiatry, 1966, 123:121-142.
Solms, M., Dreaming and REM sleep are controlled by different brain mechanisms, in Sleep and Dreaming: Scientific Advances and Reconsiderations, Edward F. Pace-Schott, Mark Solms, Mark Blagrove, and Steven Harnad, editors, Cambridge, UK, Cambridge University Press, 2003, pages 51-58.
Van de Castle, Robert L., Our Dreaming Mind, New York, Ballantine Books, 1994, pages 264, 267.

I claim:

1. A method and device for more effectively addressing the mental immune system, in humans represented by dreams, than that process as naturally evolved, the content of which can be beneficially influenced to meet particular cognitive, psychological, emotional, and behavioral requirements, consisting of a sonic apparatus that allows short, simple, and repeated questions in the dreamer's own voice to be digitally recorded and played back at a low register during preset intervals throughout the sleepstate, thus subconsciously and recurrently focusing the subject matter of dreams to desired homeostatic ends.

2. The means of claim 1 whereby said device can employ a volume control in lieu of vocal modulation by the individual speaking.

3. The means of claim 1 whereby said device may be configured in an audiocassette recording and playback format.

4. The means of claim 1 whereby said device could be utilized in conjunction with an apparatus for producing visual stimuli affecting the dreamstate.
